(19) United States
(12) Patent Application Publication
(10) Pub. No.: US 2007/0275085 A1
(43) Pub. Date: Nov. 29, 2007

(54) Compositions and methods for the cure of HIV / AIDS

(76) Inventor: Rajesh Radhakrishnan, Brampton (CA)

Correspondence Address: RAJESH RADHAKRISHNAN, 20 MORADO COURT, BRAMPTON, ON, L6S 4H7, US

(21) Appl. No.: 11/713,817

(22) Filed: Mar. 5, 2007

(30) Foreign Application Priority Data
May 26, 2006 (CA) PCT/CA06/00867
Dec. 4, 2006 (CA) PCT/CA06/01970

(51) Int. Cl. A61K 36/58; A61K 35/20

(52) U.S. Cl. 424/535; 424/761

(57) ABSTRACT

A pharmaceutical composition for the treatment of patients with human immunodeficiency virus infection is disclosed. The composition comprises effective amounts of: components obtained from Neem (Azadirachta Indica); and a protein supplement. This composition can be produced by: contacting powdered Neem with water to allow said water to extract components from said Neem powder to produce an aqueous extract; and mixing the aqueous extract with a protein supplement to form a drink. A method for treating a patient with human immunodeficiency virus infection is also disclosed. This method can comprise the step of orally administering to said patient the drink.

### FIELD OF THE INVENTION

The present invention relates to the treatment of persons with human immunodeficiency virus (HIV) infections.

### BACKGROUND OF THE INVENTION

The Neem tree (Azadirachta Indica) is a plant that grows wild in many parts of the world where the climate is warm, notably, India and Africa. Throughout India and Africa, different parts of the Neem tree, inclusive of leaves, bark, flower and seed, are employed as remedies for various human ailments.

The prior art suggests the utilization of compounds derived from the Neem tree for the treatment of persons with human immunodeficiency virus (HIV) infection, which is known to be a difficult infections to treat, due in part to the ability of the virus to position itself deep within the muscle tissues of those infected.

For example, in U.S. Pat. No. 5,370,873 (Udeinya), issued Dec. 6, 1994, it is taught to produce a pharmaceutical preparation based on Neem leaves. Udeinya teaches the collection of fresh green leaves from mature trees. The leaves are oven-dried, ground to a coarse powder and extracted using a mixture of acetone and distilled water. The solvent is removed through a process of rotor-evaporation. Precipitates on the side of the rotor flask are removed with acetone. The acetone solution is allowed to evaporate at room temperature, leaving a substance designated as IRAB. IRAB is suggested to be useful to inhibit development of the HIV virus when mixed with a physiologically acceptable carrier. However, Udeinya fails to disclose dosage regimes or levels, and fails to disclose a useful carrier.

### SUMMARY OF THE INVENTION

A pharmaceutical composition for the treatment of patients with human immunodeficiency virus infection forms one aspect of the invention. This composition comprises effective amounts of: components obtained from Neem (Azadirachta Indica); and a protein supplement.

A pharmaceutical composition for the treatment of patients with human immunodeficiency virus infection forms another aspect of the invention. This composition is produced by: contacting powdered Neem (Azadirachta Indica) with water to allow said water to extract components from said Neem powder to produce an aqueous extract; and mixing the aqueous extract with a protein supplement.

A method for treating a patient with human immunodeficiency virus infection forms yet another aspect of the invention. This method comprises the step of orally administering to said patient an effective amount of components of Neem in combination with a protein supplement.

Other advantage, features and characteristics of the present invention will become more apparent upon consideration of the following detailed description and the appended claims with reference to the photograph, the latter being briefly described hereinbelow.

### BRIEF DESCRIPTION OF THE PHOTOGRAPH

FIG. 1 is a photograph demonstrating the effects of treatment.

### DETAILED DESCRIPTION

A preferred method for the treatment of HIV infection is hereinafter described.

In this method, 1.25 litres of potable water is mixed with 20 grams of commercially available powdered Neem, of the type derived from bark, leaves, flowers and seeds, to form a slurry.

The volume of the slurry is reduced, through boiling, to 750 ml., to extract components from the powdered Neem.

The reduced slurry is filtered through cloth to produce an aqueous extract containing components extracted from the Neem powder and to also produce a residue of depleted Neem powder.

The aqueous extract is mixed with 112.5 grams of a commercially available protein drink mix powder to produce a pharmaceutical composition in drink form. A serving of the drink is ingested by a patient 5 times daily, 5-6 days per week, such that the total amount of protein ingested by the patient is about 1 gram/kilogram body weight/day. In this preferred method, the protein drink mix powder is of the type including about 89 wt. % cross-flow microfiltered Whey protein Isolate, with the remaining 11 wt. % comprising Corosolic Acid, Protease I, Protease II, Peptyzyme SP, lactase, Amylase, Sucralose and Piperine Extract. For example, in the case of a 100 kg Patient, each serving is prepared by mixing 150 ml aqueous extract with 22.5 grams protein drink mix powder (which contains approx. 20 grams protein).

In the event of extreme fatigue, treatment is arrested for 2-3 days and then resumed.

Treatment in this manner continues for at least one year.

Thereafter, once blood HIV viral counts have fallen below 50/ml, treatment continues, but the amount of Neem in each serving is cut in half.

Thus, in the case of the preferred method described hereinbefore, 10 grams of powdered Neem, rather than 20 grams, is used to form the slurry.

Thereafter, the treatment continues for atleast one year and until skin discoloring appears on limb nodes and other parts of human body.

Thus, in the case of the preferred method described hereinbefore, protein suppiement is mixed with water, rather than aqueous Neem extract to form a drink.

The treatment continues until the HIV infection is eradicated, which may take 2-3 years.

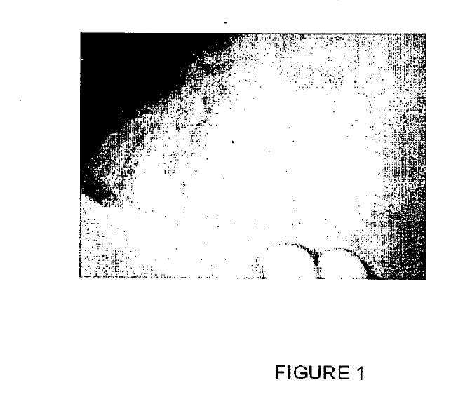

FIG. 1 is a photograph of the skin of a male patient in the course of treatment. The visible tissue discoloration within encircled area 20 is evidence of the composition at work eradicating the HIV virus deep within the muscle tissues of the patient.

Without intending to be bound by theory, it is believed that the components extracted from Neem are adapted to facilitate the production of antibodies against the HIV virus. As the primary constituent of antibodies is protein, this production is supported by the protein supplement, which makes protein readily available in the body for antibody production.

While but a single embodiment of the present invention has been herein shown and described, it will be understood that various changes may be made. For example, whereas the use of a commercially available protein drink mix supplement is taught, other sources of protein could be utilized. Simple Whey protein isolate could, for example, be utilized in the place of the protein drink mix supplement. As well, whereas the water used to extract components from powdered Neem is described to be used to form the basis of a drink, the water could equally be evaporated from the aqueous extract, to leave a Neem residue which could be mixed with protein and formed into a tablet, capsule or the like, for daily ingestion, or formed into a powder for subsequent mixture with water to form the drink. The drink itself could also be dehydrated and powdered, for subsequent rehydration and use at a later date. The protein supplement and Neem components could also be ingested separately. Whereas Whey Protein Isolate is taught for use as the protein supplement, other protein sources could be utilized. Compounds used by the body in protein formation, i.e. amino acids, could also be utilized, and are intended to fall within the meaning of the term “protein supplements” as utilized herein. Accordingly, the invention should be understood as limited only by the claims appended hereto, purposively construed.

1. A pharmaceutical composition for the treatment of patients with human immunodeficiency virus infections, said composition comprising effective amounts of:
components obtained from Neem (Azadirachta Indica); and
a protein supplement.

2. A pharmaceutical composition according to claim 1, wherein the components are obtained by extracting powdered Neem with water.

3. A pharmaceutical composition according to claim 1, wherein the protein supplement comprises of about 89% by weight of whey protein isolate and about 11% by weight of pro performance enzyme complex (protease I, protease II, peptizyme SP, lactase, amylase), sucralose, corosolic acid and piperine extract.

4. A pharmaceutical composition according to claim 1, wherein the components are obtained from the bark, seed, flower and leaf of Neem.

5. A pharmaceutical composition according to claim 1 in the form of drink.

6. A pharmaceutical composition for the treatment of patients with human immunodeficiency virus infections, said composition being produced by:
contacting powdered Neem (Azadirachta Indica) with water to allow said water to extract components from said Neem powder to produce an aqueous extract; and
mixing the aqueous extract with a protein supplement.

7. A composition according to claim 6, wherein the powdered Neem is powder of Neem bark, seed, flower and leaf.

8. A composition according to claim 6, wherein the contacting step comprising boiling the water.

9. A composition according to claim 8, wherein about 40% of the water volume is evaporated during the contacting step.

10. A composition according to claim 6, wherein the water is contacted with the powdered Neem and thereafter filtered to produce the aqueous extract and a residue of depleted Neem powder.

11. A composition according to claim 6, wherein the powdered Neem and water are provided in the weight ratio of about 20:1250 for the contacting step.

12. A composition according to claim 6, wherein the aqueous extract and the protein supplement are mixed in a weight ratio of about 1500:225 in the mixing step.

13. A method for treating a patient with human immunodeficiency virus infections, said method comprising the step of orally administering to said patient an effective amount of components of Neem in combination with a protein supplement.

14. A method according to claim 13, wherein the effective amount of components of Neem and the protein supplement are provided in the form of a drink produced by:
contacting powdered Neem (Azadirachta Indica) with water to allow said water to extract components from said Neem power to produce an aqueous extract; and
mixing the aqueous extract with a protein supplement.

15. A method according to claim 14, wherein the powdered Neem is powder of Neem bark, seed, flower and leaf.

16. A method according to claim 14, wherein the contacting step comprising boiling the water.

17. A method according to claim 14, wherein the water is contacted with the powdered Neem and thereafter filtered to produce the aqueous extract and a residue of depleted Neem powder.

18. A method according to claim 14, wherein the aqueous extract and the protein supplement are mixed in a weight ratio of about 1500:225 in the mixing step.

19. A method according to claim 14, wherein, the initial treatment step,
in the drink, the weight ratio of:
the grams of powdered Neem from which the components of Neem are derived;
to
protein
is about 1:5 and
⅕ of the total quantity of drink for a day is orally administered every three hours daily to the patient, such that the aggregate amount of the protein administered to the patient each day is 1 gram per kilogram of the body weight; and
for at least one year and until blood viral counts fall below 50 per ml.

20. A method according to claim 19, wherein, after the initial treatment step, the second treatment step,
in the drink, the weight ratio of:
the grams of powdered Neem from which the components of Neem are derived;
to
protein
is about 1:10 and
⅕ of the total quantity of drink for a day is orally administered every three hours daily to the patient, such that the aggregate amount of the protein administered to the patient each day is 1 gram per kilogram of the body weight; and
for at least one year and until skin discoloring appears on limb nodes and other parts of human body.

21. A method according to claim 20, wherein, after the second treatment step,
⅕ of total grams of protein supplement (whey protein isolate) for a day is administered orally in form of drink every three hours daily to patient, such that aggregated amount of protein administered to patient each day is 1 gram per kilogram of body weight; and
until the host cells infected with HIV infections are eliminated.
